(19) United States
(12) Patent Application Publication
(10) Pub. No.: US 2009/0297403 A1
(43) Pub. Date: Dec. 3, 2009

(54) METHOD FOR PRODUCING A BIOREACTOR OR LAB-ON-A-CHIP SYSTEM AND BIOREACTORS OR LAB-ON-A-CHIP SYSTEMS PRODUCED THEREWITH

(76) Inventors: Volker Franke, Dresden (DE); Frank Sonntag, Dresden (DE); Jan Hauptmann, Dresden (DE)

Correspondence Address: JACOBSON HOLMAN PLLC, 400 SEVENTH STREET N.W., SUITE 600, WASHINGTON, DC, 20004, US

(21) Appl. No.: 12/310,319

(22) PCT Filed: Aug. 29, 2007
(86) PCT No.: PCT/DE2007/001578

§ 371 Date: Jul. 15, 2009

(30) Foreign Application Priority Data
Aug. 31, 2006 (DE) 10 2006 040 773.3

(51) Int. Cl. B32B 37/04; C12Q 1/00

(52) U.S. Cl. 422/99; 156/272.2; 156/272.8

(57) ABSTRACT

The invention relates to a method for the manufacture of a bioreactor or of a lab-on-a-chip system as well as to bioreactors or lab-on-a-chip systems manufactured therewith. In this respect, at least two different components are connected to one another. It is the object of the present invention to set forth a method with which bodies having very different melting points, namely a ceramic material and a polymer, can be connected to one another independently of whether the surfaces to be connected are accessible from the outside or not. In the method in accordance with the invention, a first body made from a polymer which is at least partially transparent for electromagnetic radiation of at least one wavelength λ, and a second body made from a ceramic material which absorbs electromagnetic radiation of the at least one wavelength λ are connected to one another. The first body is at least regionally meltable. In a first step, the first body and the second body are arranged contacting one another while forming contact surfaces such that the body is meltable in at least one region of its contact surface to the other body. In a second step, the at least one meltable region of the contact surface is brought to melting in that electromagnetic radiation of the wavelength λ is irradiated through the first body onto the meltable region of the contact surface.

The invention relates to a method for the manufacture of a bioreactor or of a lab-on-a-chip system as well as to bioreactors or lab-on-a-chip systems manufactured therewith. In this respect, at least two different components are connected to one another, with the two components first being brought into contact with one another and then one of the components thereby being melted at its contact surface to the other component. In the course of this, electromagnetic radiation is radiated through one of the components onto the contact surface.

In accordance with the prior art, technologies are known for the connection of bodies, on the one hand, in which the bodies to be connected are adhesively bonded to one another. An adhesive is introduced between the two bodies to be connected in this process and the bond is subsequently fixed e.g. by curing the adhesive. A substantial disadvantage of the adhesive bonding is that an additional material has to be introduced into the system to be connected which under certain circumstances has unwanted effects on the function of the finished component.

It is furthermore known in accordance with the prior art to weld components to one another. For this purpose, both components are melted at their surfaces to be connected. The melted regions of the components intermingle and present a fixed connection after curing. It is problematic with welding, on the one hand, that the components have to be brought into connection with one another as long as the surfaces are melted. This is in particular relevant in welding using an arc or a flame if the surfaces are not accessible from the outside in the connected state. It is also a substantial disadvantage of welding that both bodies have to be melted. Bodies whose melting points are very different cannot be connected by welding if the melting temperature of the body melting at a higher temperature is above that temperature at which the body melting at a colder temperature starts to decompose.

It is thus e.g. necessary in the manufacture of lab-on-a-chip systems or of bioreactors to be able to carry out an optical detection in the interior from the outside. Optically transparent windows are required for this. They have previously been fastened to a ceramic body by an adhesive bond; however, the disadvantages already named above have to be take into account. Adhesive bonds are, however, as a rule not tight in the long term, which is, however, required in the articles to be manufactured in accordance with the invention. Such a possibility has been described by W. Smetana et al. in “Set-up of a biological monitoring module realized in LTCC technology”; SPIE Photonics West; San Jose; Jan. 20-25, 2007.

It is therefore the object of the present invention to set forth a method with which bodies having very different melting points, namely a ceramic material and a polymer, can be connected to one another independently of whether the surfaces to be connected are accessible from the outside or not.

This object is satisfied by the method in accordance with claim 1 and by bioreactors or lab-on-a-chip systems manufactured therewith in accordance with claim 20. Advantageous further developments of the method, of the apparatus and of the bioreactor are given in the respective dependent claims.

The method in accordance with the invention has the underlying idea of connecting two bodies by melting while they are in contact with one another. In this process, one of the two bodies to be connected which is made from a polymer is irradiated by electromagnetic radiation of a specific wavelength λ, whereas the other body made from a ceramic material absorbs electromagnetic radiation of the same wavelength λ. The two bodies to be connected are first brought into contact with one another and the electromagnetic radiation is subsequently radiated onto the interface between the two bodies through the body transparent for the corresponding wavelengths of the electromagnetic radiation. The electromagnetic radiation is absorbed by the other body and thus results in a heating of the interface. In the present case, the melting point of the two bodies lies in very different regions. Only one of the two bodies, namely the body made of polymer, is melted by the irradiation of the electromagnetic radiation. The respective meltable body does not necessarily have to be meltable as a whole; it is sufficient if it is meltable in that region in which a connection to the respective other body should be established. The absorption capability and the transparency of the two bodies only has to be present for those wavelengths at which the heating of the interface should be carried out. The absorption behavior or the transmissibility at other wavelengths does not play any role. It is in particular possible to connect more than two bodies to one another. A larger number of bodies can thus e.g. be stacked over one another. The wavelength for the establishing of a specific connection between two of these bodies is then selected such that the body disposed behind the interface to be connected in the direction of incidence of the radiation absorbs the corresponding radiation, while all the bodies disposed before the interface in the direction of incidence of the ray are transparent for the radiation.

The method in accordance with the invention is particularly suitable to connect the at least two bodies to one another which cannot both be melted together. In this respect, it is particularly advantageous if the surface of that body made from a ceramic material which does not melt during the connection is roughened or structured at the contact surface to the body made from polymer to be melted. The roughening can, for example, take place by means of a laser beam or by means of sand paper. Files or other mechanical influences such as water blasting or sandblasting or milling or also chemical etching methods are also possible. What is decisive is that recesses and structures in the micrometer range can be produced in the surface. The use of a laser, advantageously of a pulsed laser, for the structuring is, however, particularly advantageous because a targeted structure can hereby be realized. Depending on the application area of the finished product, the structures can be in the order of magnitude of some micrometers or of some millimeters. Grooves or holes can e.g. be considered as the form of the structure. The grooves can, for example, have a triangular cross-section, with the tip of the triangle being able to be oriented toward the surface or in the direction of the body. Grooves with rectangular cross-sections or round cross-sections, in particular circle sectors, are also possible. In the case of a structuring by holes, the holes can be of pyramid shape, with the tips of the pyramids being able to be oriented toward the surface or into the body. In the first case, the pyramid-shaped hole would have a small opening at the surface. The recesses can also be introduced at a shallow angle to the surface.

An extremely solid connection between the melting body and the non-melting body can be achieved by such a structuring of the surfaces of the non-melting body made of ceramic material. What is important in this respect is that the melted polymer of the meltable body flows into the structures of the surface of the non-meltable body made of ceramic material and subsequently solidifies there. The meltable body can so-to-say hook into the non-meltable body in this manner.

The method described above can also be realized without a direct structuring of the surface of the non-meltable body. In this case, the melted material flows into the surface roughness portions of the non-meltable body present from the start. It is, however, advantageous both in the case of a previous structuring and in the case of a connection of non-structured bodies if the two bodies are pressed toward one another during the melting state of the surface of the meltable body. This pressing can take place, for example, by means of any desired mechanical apparatus, such as brackets, screws or clamps, but preferably takes place using a pneumatic and/or hydraulic press or a press made in a different manner. A pressure of 1 bar is particularly good for the connection of, for example, a meltable polymer to a ceramic material. Depending on the size and shape of the surface structures, on the material properties of the bodies to be connected and on the gap between them, however, a higher or lower pressure can also be applied. It is decisive, on the one hand, that the melted material is pressed into the surface structures of the non-melting body made of ceramic and, on the other hand, that the heat conduction between the bodies is sufficiently large to effect a melting. Due to the very small thermal conductivity of the ceramic material, the heat conduction takes place in this connection almost exclusively in the region in which the actual connection of the two bodies should be established and in which the electromagnetic radiation is effective.

The pressure can furthermore also be applied spotwise, for example by a sliding or rolling welding head. The latter can be designed such that it brings the electromagnetic radiation to the jointing point simultaneously with the pressing.

In accordance with the invention, a large number of different materials can be connected to one another. The described method is particularly suitable for the connection of ceramic materials to thermoplastic polymers.

The electromagnetic radiation for the melting of the meltable body made of polymer can be generated in various manners. The use of a laser, advantageously of a continuous laser, is particularly advantageous. Its wavelength can be in the visible range and/or in the near infrared range and/or in the far infrared range. A wavelength between 800 nm and 1090 nm is particularly suitable for the connection of ceramic material to a thermoplastic. The power of the laser is selected such that the desired temperature is adopted during the absorption in the boundary region. It is, however, also possible, to generate the electromagnetic radiation by means of a sufficiently powerful incandescent lamp.

In a further advantageous embodiment, at least one contact surface of the two bodies can be activated at least in part by a suitable treatment. Basically, all conventional measures for the surface activation of solid bodies are suitable for this purpose, but the activation preferably takes place chemically or energetically. Etching processes or surface derivatization e.g. with reactive compounds can e.g. be considered as the chemical activation processes; in particular radiation processes, preferably using ultraviolet radiation, can be considered as energetic activation. Already previously named mechanical measures are generally also suitable for the roughening or structuring for this purpose.

The main advantage of the method in accordance with the invention is that materials with very different melting points can be connected to one another. Meltable bodies can furthermore also be connected to such bodies which decompose on heating such as thermosetting plastics.

The bioreactors or lab-on-a-chip systems manufactured in accordance with the invention have at least one processing region containing or consisting of ceramic material. The processing region is closed at at least one side by a transparent window comprising polymer or thermosetting plastic. The transparent window is connected to the processing region by the method in accordance with the invention.

The processing region can have at least one plate-shaped divider which can be arranged parallel next to the at least one transparent window while sealingly contacting it. The divider divides the processing region into at least one compartment. At least two dividers for the formation of a plurality of compartments can also be present.

It is a further advantage that no additives have to be used for the connection, whereby impairments to the function of the connected component can be avoided.

Very solid connections can be established by the method in accordance with the invention without a material conversion taking place. Almost no mechanical strains occur in the joining region, even with temperature change strain.

The method in accordance with the invention will be explained in detail with reference to some examples in the following. There are shown

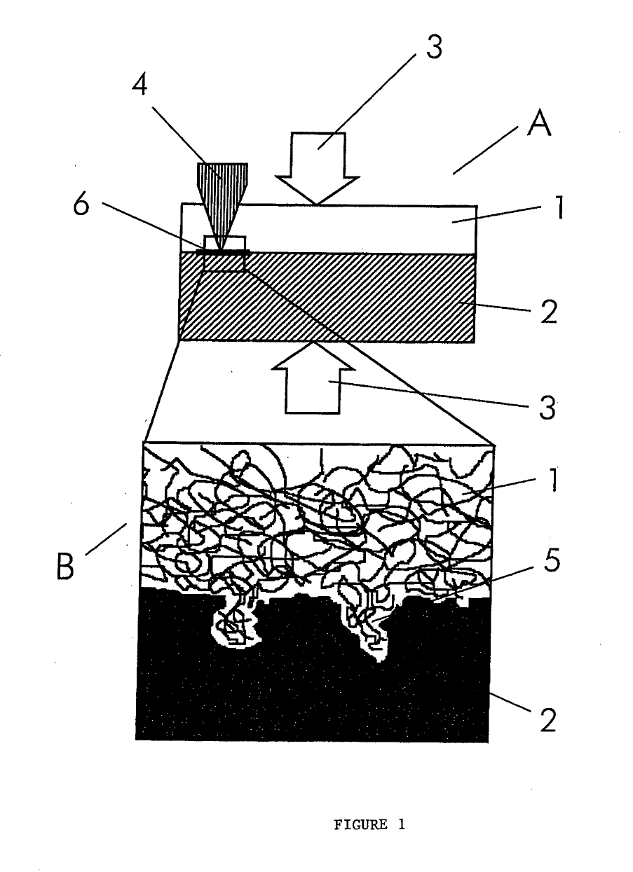

FIG. 1 the principle of the method in accordance with the invention;

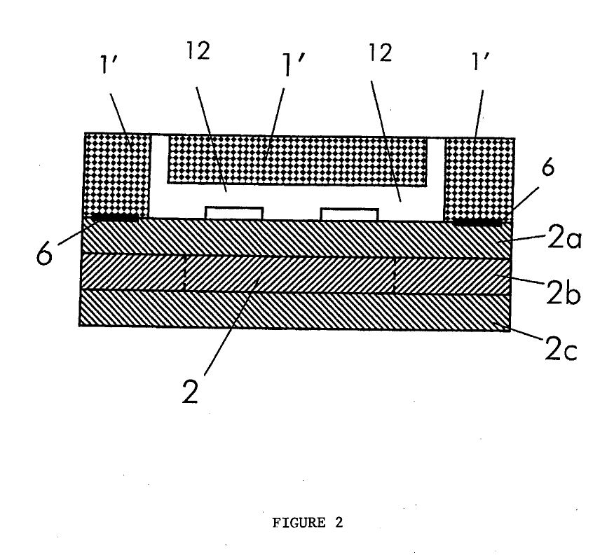

FIG. 2 a layer system manufactured by means of the method in accordance with the invention; and

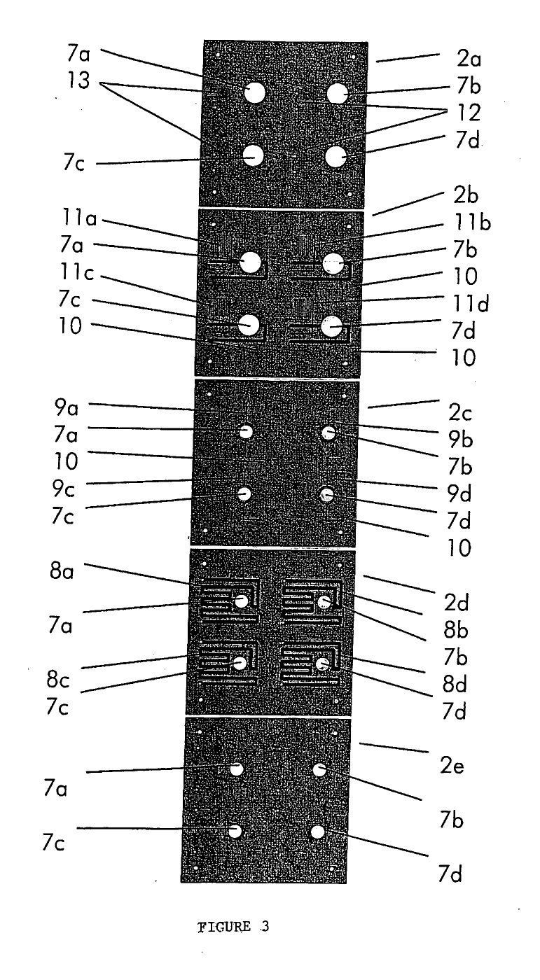

FIG. 3 a number of layers to be connected of a bioreactor which can be closed by an optically transparent window with the help of the method in accordance with the invention.

FIG. 1 shows the principle of the method in accordance with the invention. In this respect, A shows a total view and B an enlargement of the boundary region between the bodies 1 and 2. In the example shown, a meltable body 1 made from a polymer is connected to a body 2 made from a ceramic material and not meltable at the same temperature. The body 1 is first arranged contacting the body 2 and the two bodies 1 and 2 are pressed toward one another at a pressure 3. Electromagnetic radiation 4 is now irradiated through the melting body 1 onto the non-melting body 2. It is important here that the meltable body 1 is transparent for electromagnetic radiation 4 of the given wavelength whereas the non-melting body 2 is not transparent for the electromagnetic radiation of this wavelength, but rather absorbs it. The transparency of the melting body 1 for electromagnetic radiation 4 of the irradiated wavelength does not have to be one hundred percent, it only has to be so large that the meltable body 1 does not already melt itself due to the absorption of the irradiated radiation. The degree of absorption of the body 2 not meltable at the given temperature accordingly only has to be so large that a sufficient amount of heat is produced at the interface between the two bodies for the melting temperature of the melting body 1 to be reached.

The enlargement B of FIG. 1 shows an idealized representation of the boundary region 6 between the meltable body 1 and the non-melting body 2. It can be recognized that the non-melting body 2 is provided with recesses 5. Considered over the total surface, these recesses 5 represent a roughening or structuring. The diameter of these recesses is, for example, in the micrometer range or in the millimeter range. If electromagnetic radiation is now irradiated through the transparent body 1 onto the non-transparent non-melting body 2, the non-melting body 2 absorbs the electromagnetic radiation 4 and heats the boundary region 6 between the two bodies. The meltable body 1 is hereby melted and its material flows into the recesses 5 in the non-meltable body 2. If the radiation of the electromagnetic radiation is ended, the interface cools, the material of the meltable body 1 hardens and hooks this body in the recesses 5 in the non-melting body 2.

FIG. 2 shows the cross-section through a layer system which was manufactured by means of the method in accordance with the invention. Such a layer system can, for example, be a lab-on-a-chip system for the analysis of cell growth under defined conditions or it can be a microbiological reactor. Such a bioreactor has a plurality of layers 2a, 2b, 2c of low temperature cofired ceramics (LTCC). They are connected via boundary regions 6 to transparent windows made of polystyrene 1′. Different media can be conducted through the bioreactor through microchannels 12. The topmost layer of the layer system made of LTCC 2a was connected to the polystyrene window 1′ in the method in accordance with the invention. For this purpose, the LTCC layers 2a, 2b, 2c were first assembled completely and sintered. The surface region was then directly structured using a pulsed Nd:YAG laser. The polystyrene window 1′ was subsequently pressed toward the topmost LTCC layer 2a at the connection point in the boundary region 6 and the melting was then carried out using a continuous laser beam 4. The surface of the reactor chamber made from LTCC was first structured for the establishing of the connection. For this purpose, on average seventeen craters per mm² were made randomly distributed at the surface. The manufacture of the craters was done using a pulsed laser with a pulse frequency of 10 kHz and pulse lengths of approx. 100 ns at a mean laser power (pulsed) of 20 watts. Around 10 pulses were irradiated per crater. In addition to the named crater structures, structures of parallel fine lines as well as combinations of craters and lines were manufactured. Subsequently to the surface structuring, the polystyrene window 1′ was connected to the body 2, as a cell reactor made of LTCC ceramic material, by irradiation of electromagnetic radiation 4. For this purpose, a laser having the wavelength 1064 nm and a laser power (cw) of 45 watts is moved over the connection point at a speed of 15 mm/s. The window 1′ made from thermoplastic polymer was pressed against the reactor chamber of LTCC at a pressure in the joining zone of 1.4 bar (60 N on 4.2 cm²).

A window 1′ can, however, also simultaneously or solely be a functional element. With a functional element and/or a window 1′, a microfluid system having microfluid elements, e.g. channels which can in turn have inlet openings and outlet openings, can also be formed between the functional element/window 1′ and the body 2.

The part elements made of LTCC 2a, 2b and 2c were connected to one another to form the reactor chamber by sintering.

FIG. 3 shows the different layers 2a, 2b, 2c, 2d, 2e of an LTCC multilayer system with five layers. Each layer contains 4 identical sub-units of the lab-on-a-chip system or bioreactor. All the layers contain large circular openings 7a, 7b, 7c, 7d which form the cell reactors when the LTCC layers 2a, 2b, 2c, 2d, 2e are layered over one another. Meander-like channels 8a, 8b, 8c, 8d are introduced at the base of the LTCC layer 2d and a temperature controlled liquid can flow through them to be able to establish a constant temperature within the cell reactor. The layer 2c disposed thereabove has LTCC-based sensors 9a, 9b, 9c, 9d with which e.g. the impedance and temperature can be measured. Passage holes 10 represent an electrical connection of the sensors to the layers disposed thereabove. An impedance measurement is used e.g. to examine changes in the cell growth, e.g. the adsorption of cells on a surface. It thereby becomes possible to analyze the reaction of a cell culture on different test media or growth conditions. Various media can be introduced through the microchannels 11a, 11b, 11c, 11d into layer 2b. Two respective channels having a large cross-section are used to supply culture medium continuously to the cells, while the narrower, meander-like channels are used for the supply of test media. The meander-like structure opens up the possibility of mixing two different test liquids with one another or of carrying out a dilution. The lab-on-a-chip system can be connected to the required supply devices and electronic measuring devices via the passage holes 13 of the topmost layer 2a. The individual layers 2a to 2e of the non-sintered ceramic are cut and structured with the help of a pulsed layer system. The layers are then stacked onto one another and sintered. The bioreactor can be hermetically closed by a window 1′ made of polystyrene with the help of the joining method in accordance with the invention described above.

1. A method for the manufacture of a bioreactor or of a lab-on-a-chip system, wherein
a first body (1, 1′) made from a polymer which is at least partially transparent for electromagnetic radiation (4) of at least one wavelength λ;
and a second body (2) made from a ceramic material which absorbs electromagnetic radiation (4) of the at least one wavelength λ; and
wherein the first body (1, 1′) is meltable at least regionally, characterized in that,
in a first step, the first body (1, 1′) and the second body (2) are arranged contacting one another while forming contact surfaces such that the body (1, 1′) is meltable in at least one region of its contact surface to the other body (2); and,
in a second step, the at least one meltable region of the contact surface is brought to melting in that electromagnetic radiation (4) of the wavelength λ is irradiated through the first body (1, 1′) onto the meltable region of the contact surface.

2. A method in accordance with the preceding claim, characterized in that the first body (1, 1′) and the second body (2) are pressed toward one another during and/or after the second step.

3. A method in accordance with claim 1, characterized in that the first body (1, 1′) and the second body (2) are pressed toward one another during and/or after the second step at a pressure of 1 bar or at a pressure greater than 1 bar.

4. A method in accordance with claim 1, characterized in that the first body (1, 1′) and the second body (2) are pressed toward one another by a mechanical device.

5. A method in accordance with the preceding claim, characterized in that the mechanical device is selected from the group comprising pneumatic presses and/or hydraulic presses.

6. A method in accordance with claim 1, characterized in that an LTCC ceramic material is used for the second body (2).

7. A method in accordance with claim 1, characterized in that the second body (2) is made from a plurality of layers (2a to 2e).

8. A method in accordance with claim 1, characterized in that the first body (1, 1′) comprises or consists of a polymer and/or a thermoplastic.

9. A method in accordance with claim 1, characterized in that, before the first step, the first body (1, 1′) and/or the second body (2) is/are roughened and/or structured at least regionally at its contact surface to the respective other body.

10. A method in accordance with claim 1, characterized in that, before the first step, the second body (2) is roughened and/or structured at least regionally at its contact surface to the first body (1, 1′).

11. A method in accordance with claim 9, characterized in that the first body (1, 1′) and/or the second body (2) is/are roughened or structured with structures in the micrometer range.

12. A method in accordance with the preceding claim, characterized in that the structures are holes and/or grooves.

13. A method in accordance with claim 9, characterized in that the roughening or structuring takes place by microstructuring using a laser.

14. A method in accordance with claim 9, characterized in that the roughening or structuring takes place by rubbing the contact surface with sandpaper, using a mill and/or by blasting.

15. A method in accordance with claim 1, characterized in that the electromagnetic radiation (4) of the at least one wavelength λ is generated using a laser.

16. A method in accordance with claim 1, characterized in that the electromagnetic radiation of the at least one wavelength λ is generated using an incandescent lamp.

17. A method in accordance with claim 1, characterized in that the wavelength λ is in the visible range and/or in the near infrared range and/or in the far infrared range and/or between 800 nm and 1090 nm.

18. A method in accordance with claim 1, characterized in that at least one part region of the contact surface of at least one body (1, 1′ or 2) is activated chemically and/or energetically before the contacting arrangement.

19. A method in accordance with the preceding claim, characterized in that the energetic activation takes place by charging with ultraviolet radiation.

20. A bioreactor or a lab-on-a-chip system having at least one processing region which comprises or consists of a ceramic material and which is closed on at least one side by a transparent window (1′) and/or functional element comprising a polymer or thermoplastic,
characterized in that
the transparent window (1′) and/or functional element is connected to the processing region by a method in accordance with claim 1.

21. A bioreactor or a lab-on-a-chip system in accordance with the preceding claim, characterized in that the processing region has at least one plate-shaped divider which is arranged parallel next to the at least one transparent window (1′) while sealingly contacting it and divides the processing region into at least one compartment.

22. A bioreactor or a lab-on-a-chip system in accordance with the preceding claim, characterized-in that the processing region has at least two plate-like dividers which are arranged parallel next to one another sealingly contacting one another and parallel next to the at least one transparent window (1′) and whose at least one compartment are partly in contact with one another.
